(19) United States
(12) Patent Application Publication
(10) Pub. No.: US 2018/0247334 A1
(43) Pub. Date: Aug. 30, 2018

(54) SYSTEM FOR UPSELLING COMPRESSIBLE AND RESILIENT CONSUMABLE PRODUCTS TO FILL CONTAINER SPACE

(71) Applicant: Kimberly-Clark Worldwide, Inc., Neenah, WI (US)

(72) Inventors: Julie Ann Paveletzke, Appleton, WI (US); Scott Kenneth Usitalo, Charlevoix, MI (US); Brenda Marie Coulter, Appleton, WI (US); Christopher Peter Olson, Neenah, WI (US)

(21) Appl. No.: 15/444,727

(22) Filed: Feb. 28, 2017

(51) Int. Cl. G06Q 30/02; A61F 13/551; B65B 61/20; G06Q 10/08; G06Q 30/06

(52) U.S. Cl. CPC G06Q 30/0253; A61F 13/55105; G06Q 30/0635; B65B 61/20; G06Q 10/083; A61F 13/551

(57) ABSTRACT

An upselling system for utilizing compressible and resilient consumable products as substitutes for conventional fill material in shipping containers can include leveraging product dimensions and compressibility to identify and offer compressible and resilient products that will fit in the container with, and in particular embodiments provide shipping protection to, the already-selected item(s).

### BACKGROUND OF THE INVENTION

When customers make purchases on-line, many businesses will offer additional products for sale, such as products to ship along with the original purchase in the same package (one form of so-called “upselling”). In some examples, a determination is made as to whether the container selected to ship the consumer's original purchase has surplus volume and weight capacity to hold additional products. See, for example, U.S. 2013/0173417 to Spremulli. However, there has not been developed a system to consider the physical interaction of the original purchase with the additional “upsell” products in the shipping container, the relevance of the physical nature of the additional “upsell” products to their compatibility with the surplus volume and weight capacity, or the environmental impact of shipping the additional “upsell” products.

### SUMMARY OF THE INVENTION

The invention pertains to a system for utilizing compressible and resilient consumable products as substitutes for conventional fill material to create an upsell opportunity. The system comprises a computer processor, and a computer memory operatively coupled to the computer processor. The computer memory has disposed within it computer program instructions configured in particular embodiments for the following steps:

receiving a customer selection of one or more items to be shipped;

determining dimensions of the one or more items to be shipped;

identifying, in dependence on the determined dimensions, a shipping container to be utilized to ship the one or more items;

determining dimensions of one or more packable spaces that will be available within the shipping container along with the one or more items previously selected to be shipped;

accessing a list of items that includes compressible and resilient consumable products;

determining from the list of items one or more compressible and resilient consumable products that will fit within at least one of the one or more packable spaces; and

displaying at least one of the one or more compressible and resilient consumable products to the customer for purchase.

In certain embodiments, the system can also include:

determining a level of shipping protection needed to prevent damage to the one or more items to be shipped;

accessing a list of items that includes compressible and resilient consumable products;

determining from the list of items one or more compressible and resilient consumable products that will fit within at least one of the one or more packable spaces and that will provide the level of shipping protection determined to be needed to prevent damage to the one or more items to be shipped; and

displaying at least one of the one or more compressible and resilient consumable products to the customer for purchase.

### BRIEF DESCRIPTION OF THE FIGURES

FIG. 1A-B is a flowchart of a system of utilizing empty shipping container space to sell compressible and resilient consumable products in accordance with one embodiment of the invention.

FIG. 1C-D is a flowchart of a system of utilizing empty shipping container space to sell compressible and resilient consumable products in accordance with an alternative embodiment of the invention.

FIG. 2 illustrates an example of determining remaining packable spaces in accordance with one embodiment of the invention.

FIG. 3 illustrates an example of a compressible and resilient consumable product suitable for use in conjunction with particular embodiments of the present invention.

### DETAILED DESCRIPTION OF PARTICULAR EMBODIMENTS

In one aspect, the invention pertains to a system for utilizing compressible and resilient consumable products as substitutes for conventional fill material to create an upsell opportunity. In particular embodiments, the system includes a computer processor, and a computer memory operatively coupled to the computer processor. Each block of the flowcharts in the Figures can be implemented by computer program instructions. These computer program instructions may be provided to a processor of a computer to produce a machine, such that the instructions, which execute via the processor of the computer or other programmable data processing means, create means for implementing the functions/acts shown in the flowchart(s). “Computer” broadly refers to any kind of device that receives input data, processes that data through computer instructions in a program, and generates output data. Such computer can be a computer, a server, a cell phone, a smart tablet, or other device. The computer memory has within it computer program instructions configured to conduct various combinations of the following steps of the system of the invention.

In particular embodiments, the system includes receiving a customer selection of one or more items to be shipped (10). For example, the order may be placed by the customer via a computer, a smartphone, or other electronic process utilizing the internet. In an optional step, the system can include determining whether a shipping container is needed to ship the item (12). If a shipping container is needed (as opposed to shipping the item(s) in the factory-provided packaging), the system in particular embodiments includes the step of determining the weight and/or dimensions of the one or more items to be shipped (14). “Dimension(s)” as used herein means length, width, and/or height of the smallest rectangular parallelepiped that would entirely contain an item. The weight and dimensions of each item in the on-line store may be stored in a database. The system can further include identifying, in dependence on the determined dimensions, a shipping container to be utilized to ship the one or more items (16). The system can include a database of all available shipping containers and the volume capacity and weight capacity of each shipping container.

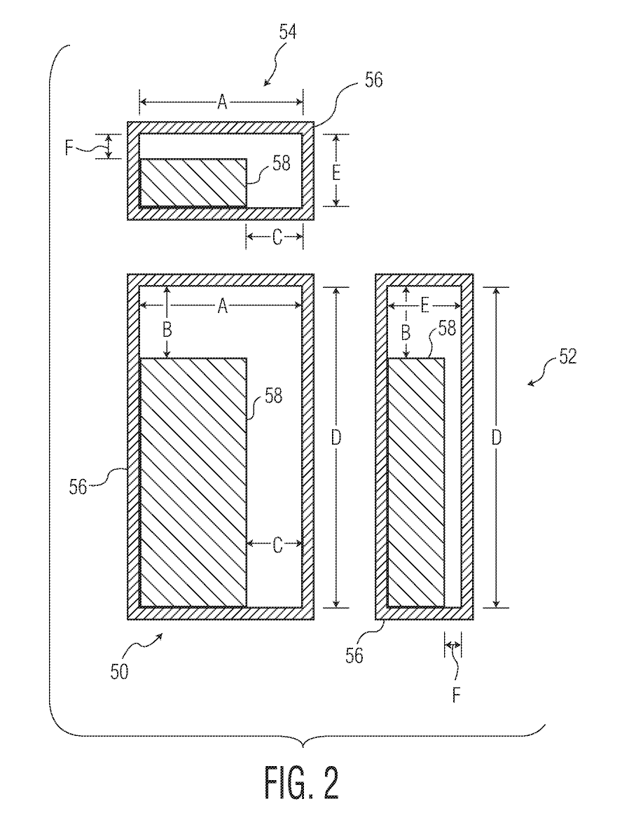

In particular embodiments, the system further includes determining dimensions of one or more packable spaces that will be available within the shipping container along with the one or more items previously selected to be shipped (18). “Packable spaces” means spaces within the remaining volume capacity into which compressible and resilient consumable products may be placed. The concept of packable spaces is illustrated in FIG. 2. FIG. 2 illustrates a top view 50, a side view 52, and an end view 54 of an item 58 to be shipped within a shipping container 56. The packable spaces are to the right of and above item 58 in the top view 50, to the right of and above item 58 in the side view, and to the right of and above item 58 in the end view. Thus, in the example of FIG. 2, there are three discrete parallelepiped packable spaces: one packable space measures A×B×E; a second packable space measures C×D×E; and a third packable space measures A×D×F.

If there are any packable spaces identified in the previous step 18 (such as any package spaces that exceed a certain volumetric threshold), the system further includes accessing a list of items that includes compressible and resilient consumable products (22) that could fill one or more packable spaces and that can be offered for sale to the customer. The list may comprise all items for sale in the on-line store, or may comprise a subset of all items for sale, such as a list or filtering that includes only compressible and resilient consumable products. From the list, each item whose weight is greater than the determined remaining weight capacity of the shipment or container is in particular embodiments removed from consideration (24). Additionally or alternatively, each item whose dimensions or minimum compressed dimensions (described below) are greater than the determined packable space dimensions is in particular embodiments removed from consideration (34). In this way, the method in particular embodiments includes determining from the list of items one or more compressible and resilient consumable products that will fit within at least one of the one or more packable space (also referred to herein as “qualifying products”).

If any items remain in the list (36) after removing non-qualifying items from consideration as described in the previous paragraph, the system can include displaying at least one of the one or more “qualifying” compressible and resilient consumable products to the customer for purchase (38). In particular embodiments, the system can include offering a qualifying product to the customer at a discounted price (such as to pass along part or all of the shipping-cost savings to the consumer), or can include offering the qualifying product to the customer for free. The discounted price or no price promotion can be funded by the manufacturer of the product, or the on-line retailer, or both.

If the customer selects one or more of the qualifying consumable products that are displayed (40), the selected product is added to the order (42). In particular embodiments, the system can also include generating instructions for filling the shipping container (not shown). If the manner in which the selected qualifying product should be included in the container along with the originally purchased one or more items 58 includes multiple possibilities, generating instructions for filling the shipping container (such as instructions to a human packer or a robotic packer) can ensure than the items are positioned in the container to fit most efficiently, or to accomplish functional objectives, such as to provide shipping protection as described below.

Note that some of the filters (such as the weight filter) may be omitted entirely in particular embodiments.

In particular embodiments, determining one or more compressible and resilient consumable products which will fit within at least one of the one or more packable spaces comprises determining one or more compressible and resilient consumable products for which each dimension is equal to or smaller than a corresponding dimension of one of the one or more packable spaces. For example, consider the packable space in FIG. 2 which measures A×B×E, with A being the largest dimension, B being the middle size dimension, and E being the smallest dimension. An item will fit within the A×B×E packable space if (1) the largest dimension of the item is less than A; (ii) if the middle size dimension of the item is less than B; and (iii) if the smallest dimension of the item is less than E.

It has been discovered, however, that even if one of the dimensions of the compressible and resilient consumable product exceeds the corresponding dimension of a packable space, the product may in certain instances still be suitable, and in some cases desirable, for inclusion in the packable space, and thus suitable for offering for sale to the customer in the transaction at hand. In particular embodiments, each of the compressible and resilient consumable products on the list of qualifying products can be compressed in at least one dimensional direction by up to a maximum percentage without damage to the product or packaging material surrounding the product to define a minimum compressed dimension. “Dimensional direction” means a direction parallel to any one of the product's length, width, or height dimensions. “Damage” means deformation that is not substantially reversible and that affects the functionality or aesthetic appearance of the product or the original product packaging. By way of example, a container of facial tissue, breakfast cereal, or toothpaste surrounded by a convention rigid cardstock carton would typically not be able to withstand compression without damage, because the cardstock carton would become creased or folded in a way that would result in irreversible functional or aesthetic degradation of the carton. Similarly, a flexible package of potato chips, bread, or plastic disposable safety razors would typically not be able to withstand compression without damage, because although the plastic packaging “bag” is flexible, the product within the flexible packaging bag would likely become smashed, cracked, or crushed in a way that would result in irreversible functional or aesthetic degradation of the product. In contrast, paper dinner napkins packaged in flexible plastic film, kitchen paper towels packaged in flexible plastic film, toilet paper packaged in flexible plastic film, or kitchen sponges packaged in flexible plastic film would typically be able to withstand compression without damage, because both the product and the flexible plastic film is flexible and/or compressible, and the product itself is substantially fully resilient after being compressed, and neither the product nor the packaging would become deformed in a way that would result in irreversible functional or aesthetic degradation of the product, so long as the compression in the relevant dimensional direction(s) did not in particular embodiments exceed some threshold.

Therefore, a database of product information includes not only the dimensions of the compressible and resilient consumable products, but also information about the products' compressibility, such as a minimum compressed dimension in one or more dimensional directions. For example, a database may list the dimensions of a parallelepiped-shaped package of dinner napkins as 12 inches long by 6 inches width by 6 inches high, but may also list a minimum compressed length of 10 inches, a minimum compressed width of 5 inches, and/or a minimum compressed height of 5 inches. In this way, a given dimension of a packable space suitable to receive a compressible product could be as low as the minimum compressed dimension of such compressible product. Said another way, a compressible and resilient consumable product may exceed in size the size of a package space in one or more dimensions, so long as the minimum compressed dimension of the consumable product does not exceed the corresponding dimension of the package space.

Accordingly, in particular embodiments, determining one or more compressible and resilient consumable products which will fit within at least one of one or more packable spaces includes determining one or more compressible and resilient consumable products for which first and second dimensions are equal to or less than corresponding first and second dimensions of one of the one or more packable spaces, and for which a minimum compressed dimension is equal to or less than a corresponding third dimension of one of the one or more packable spaces. For example, a packable space measuring 10×6×6 inches could accommodate the package of dinner napkins in the previous paragraph because although the package of napkins has uncompressed dimensions of 12×6×6 inches, the length can be compressed to a minimum compressed length of 10 inches, which is equal to or less than the 10-inch length of the packable space. Therefore, the package of dinner napkins can be included on the list of qualifying products.

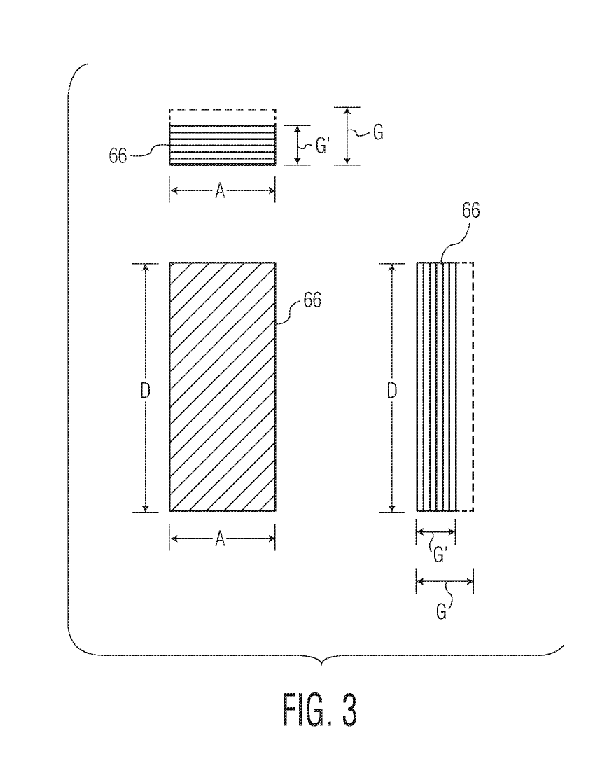

In another example, referring to FIGS. 2 and 3, the packable space measuring A×D×F (FIG. 2) could accommodate a package of dinner napkins 66 measuring A×D×G (FIG. 3), where the uncompressed thickness G of the napkins is 25 percent greater than F, if the package of dinner napkins can be compressed in the thickness dimensional direction (the “G” direction) to a minimum compressed thickness of G^{I} that is no greater than F. If the dinner napkins can be compressed to a thickness G^{I} that is no greater than F, the package of dinner napkins can be included on the list of qualifying products.

In particular embodiments, each compressible and resilient consumable product on the list of items comprises an absorbent product wrapped in a pliable polymeric film. Example of such products include consumable tissue products, personal care absorbent articles, and disposable diapers.

In particular embodiments, at least some compressible and resilient products in the database are sized so that at least two dimensions are the same as, or nearly the same as (within 5 percent) two of the inner dimensions of a shipping container commonly used by an on-line retailer. Many on-line retailers, such as Amazon.com, employ a selection of common shipping boxes. For example, one commonly sized shipping box may have the dimensions A×D×E as representatively illustrated in FIG. 2. In particular embodiments, at least some compressible and resilient products in the database are produced by the manufacturer to have the dimensions A×D×X (where X may be less than, equal to, or greater than E; A×Y×E (where Y may be less than, equal to, or greater than D); or Z×D×E (where Z may be less than, equal to, or greater than A). In this way, a selection of compressible and resilient products is available to very efficiently fill empty space in shipping containers, because they are sized to suitably fit into packable spaces. This is true even if X exceeds E, Y exceeds D, and/or Z exceeds A, because the consumable product is compressible and resilient and can in particular embodiments be compressed in the X, Y, and/or Z dimensional directions to fit into the relevant packable space.

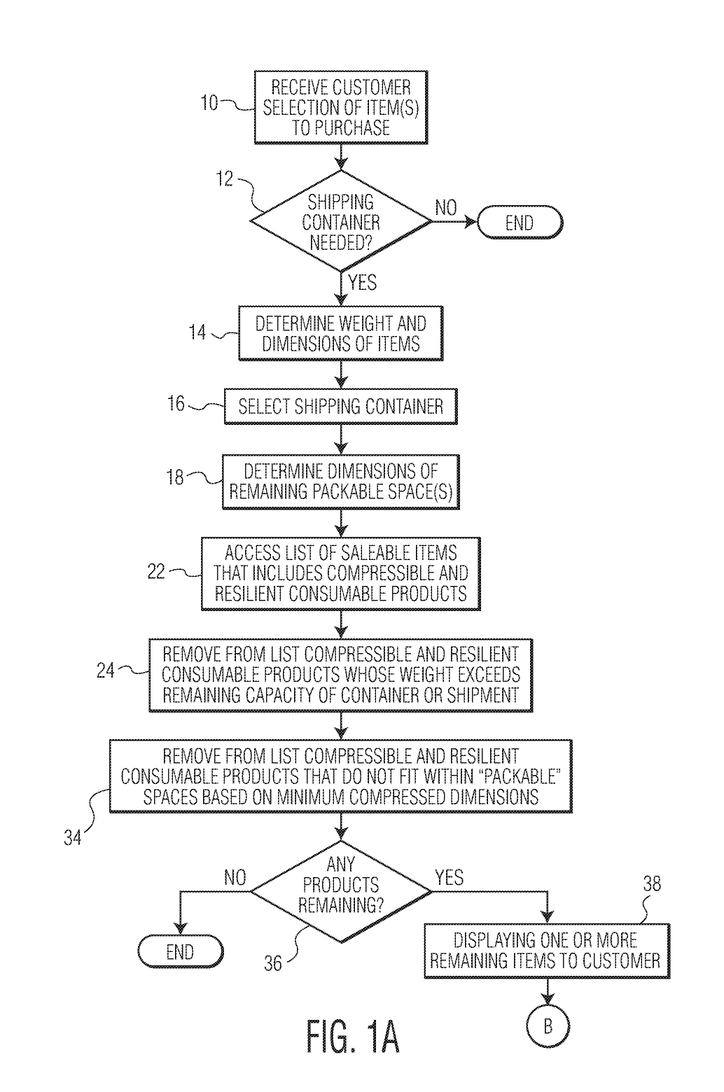

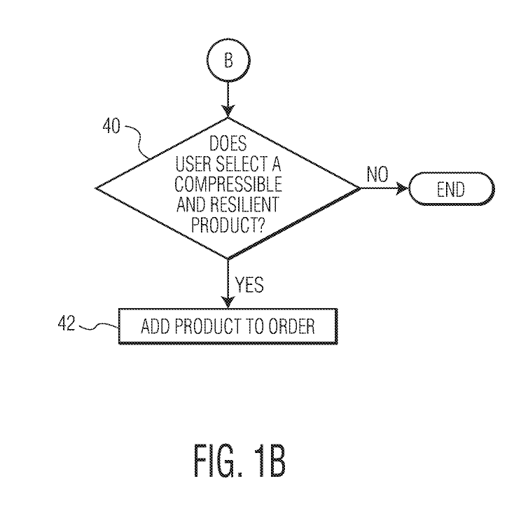

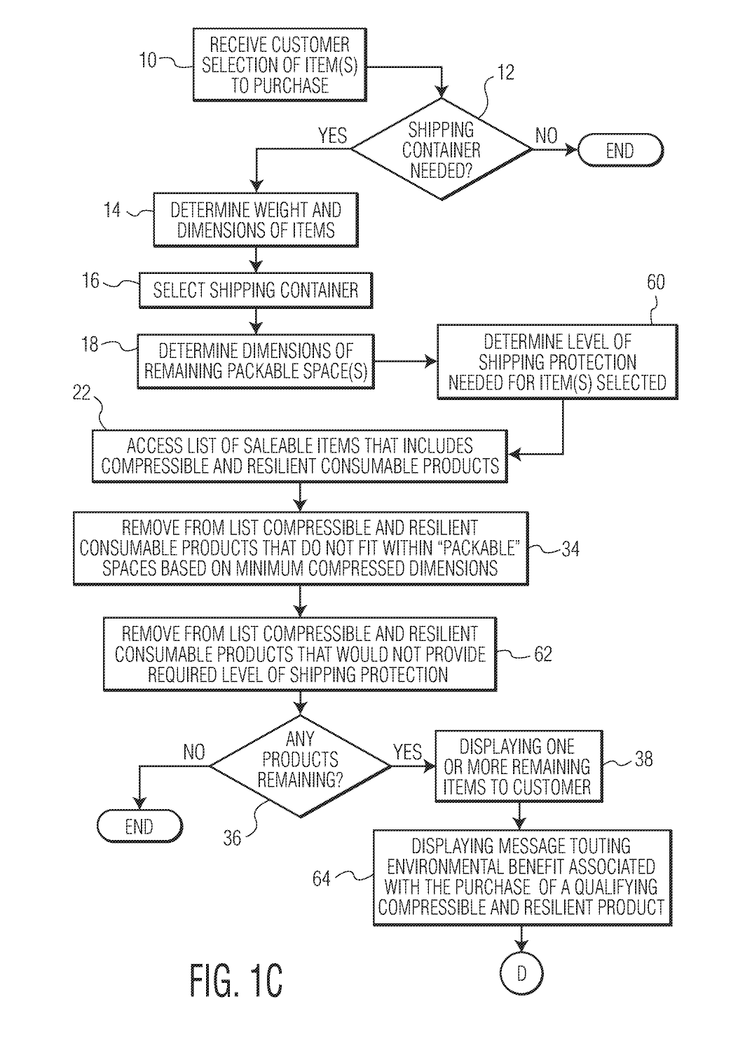

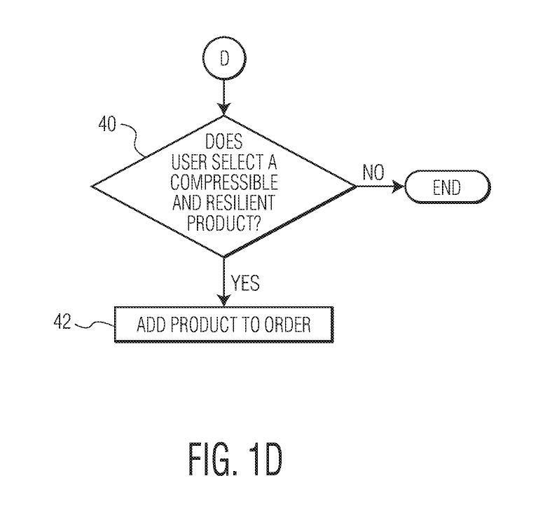

In particular embodiments, representatively illustrated in FIG. 1C, the system further includes determining a level of shipping protection needed to prevent damage to the one or more items to be shipped (60). “Shipping protection” means the amount or nature of padding (provided by, for example, compressible product or conventional filler material) necessary to be placed within a shipping container to cushion, secure, and otherwise maintain the integrity of other purchased items to be shipped in the container. Each item available for on-line purchase from the seller may be listed in a database along with information pertaining to the fragility of the item, and/or the nature of shipping protection needed. Any suitable system of grading or rating the fragility of an article, or the required level of shipping protection needed, could be used. For example, an item could be graded as being “not fragile,” “moderately fragile,” or “highly fragile.” Another grading scale could be “needs no shipping protection,” “needs moderate shipping protection,” or “needs high shipping protection.”

Furthermore, a database of product information includes not only the dimensions of the compressible and resilient consumable products, but also information about the compressible and resilient consumable products' suitability to provide a required level of shipping protection. For example, a roll of kitchen paper towels may be rated as suitable for providing a high level of shipping protection, whereas a tightly packaged bag of baby wipes may be rated as suitable for providing only a moderate level of shipping protection. Similarly, a high-loft, low-density package of toilet paper may be rated as suitable for providing a high level of shipping protection, whereas a low-loft, high-density package of toilet paper may be rated as suitable for providing only a moderate level of shipping protection.

In particular embodiments, the system further includes determining from the list of items one or more compressible and resilient consumable products that will fit within at least one of the one or more packable spaces and that will provide the level of shipping protection determined to be needed to prevent damage to the one or more items to be shipped, and removing from the list those products that would not provide the required level of shipping protection (62). The system can further include displaying this “qualifying” compressible and resilient consumable product(s) to the customer for purchase (38). In some embodiments, the selection of one or more qualifying products does not increase the shipping cost. In other embodiments, the qualifying product can increase the shipping cost, such as if the qualifying product increases the weight of the entire shipment into a higher range of shipping costs.

If the consumer decides to purchase a qualifying compressible and resilient consumable product, such as via any of the particular embodiments described above, many benefits may be obtained. First, the consumer is able to have shipped to her a consumable product at low or no additional marginal shipping expense. Second, the on-line seller is spared the expense of packing “fill” material that may have otherwise had to occupy the space occupied by the qualifying product. Third, the solid waste stream is spared the burden of the packing “fill” material that otherwise would have been used and thrown in the trash.

To highlight some of these potential benefits to the consumer and to increase the potential that she will purchase a qualifying product, in particular embodiments the system includes displaying a message touting an environmental benefit associated with the purchase of a qualifying compressible and resilient product (64). For example, the system could include displaying a quantity of conventional fill material avoided by the customer's purchase of the one or more compressible and resilient consumable products.

While the invention has been described in detail with respect to the specific aspects thereof, it will be appreciated that those skilled in the art, upon attaining an understanding of the foregoing, may readily conceive of alterations to, variations of, and equivalents to these aspects. Accordingly, the scope of the present invention should be assessed as that of the appended claims.

What is claimed is:

1. A system for utilizing compressible and resilient consumable products as substitutes for conventional fill material to create an upsell opportunity, the system comprising a computer processor, a computer memory operatively coupled to the computer processor, the computer memory having disposed within it computer program instructions configured for:
receiving a customer selection of one or more items to be shipped;
determining dimensions of the one or more items to be shipped;
identifying, in dependence on the determined dimensions, a shipping container to be utilized to ship the one or more items;
determining dimensions of one or more packable spaces that will be available within the shipping container along with the one or more items previously selected to be shipped;
accessing a list of items that includes compressible and resilient consumable products;
determining from the list of items one or more compressible and resilient consumable products that will fit within at least one of the one or more packable spaces; and
displaying at least one of the one or more compressible and resilient consumable products to the customer for purchase.

2. The system of claim 1 wherein each compressible and resilient consumable product on the list of items comprises an absorbent product wrapped in a pliable polymeric film.

3. The system of claim 1 wherein determining one or more compressible and resilient consumable products which will fit within at least one of the one or more packable spaces comprises determining one or more compressible and resilient consumable products for which each dimension is equal to or smaller than a corresponding dimension of one of the one or more packable spaces.

4. The system of claim 1 wherein each of said compressible and resilient consumable products on said list of items can be compressed in at least one dimensional direction without damage to the product or packaging material surrounding the product to define a minimum compressed dimension.

5. The system of claim 4 wherein determining one or more compressible and resilient consumable products which will fit within at least one of the one or more packable spaces comprises determining a compressible and resilient consumable product for which (1) first and second dimensions are collectively equal to or smaller than corresponding first and second dimensions of one of the one or more packable spaces; (2) a third dimension is greater than the corresponding third dimension of the one or more packable spaces; and (3) a minimum compressed third dimension is equal to or smaller than a corresponding third dimension of one of the one or more packable spaces.

6. The system of claim 1 wherein the computer program instructions are further configured for displaying a quantity of conventional fill material avoided by the customer's purchase of the one or more compressible and resilient consumable products.

7. The system of claim 1 wherein the system further includes generating instructions to a human or a robotic machine for filling the shipping container.

8. The system of claim 1 wherein a plurality of compressible and resilient products in the list of items are sized so that at least two dimensions are within 5 percent of two inner dimensions, respectively, of a shipping container commonly used by an on-line retailer.

9. A system for utilizing compressible and resilient consumable products as substitutes for conventional fill material to create an upsell opportunity, the system comprising a computer processor, a computer memory operatively coupled to the computer processor, the computer memory having disposed within it computer program instructions configured for:
receiving a customer selection of one or more items to be shipped;
determining dimensions of the one or more items to be shipped;
identifying, in dependence on the determined dimensions, a shipping container to be utilized to ship the one or more items;
determining a level of shipping protection needed to prevent damage to the one or more items to be shipped;
determining dimensions of one or more packable spaces that will be available within the shipping container along with the one or more items previously selected to be shipped;
accessing a list of items that includes compressible and resilient consumable products;
determining from the list of items one or more compressible and resilient consumable products that will fit within at least one of the one or more packable spaces and that will provide the level of shipping protection determined to be needed to prevent damage to the one or more items to be shipped; and
displaying at least one of the one or more compressible and resilient consumable products to the customer for purchase.

10. The system of claim 9 wherein each compressible and resilient consumable product on the list of items comprises an absorbent product wrapped in a pliable polymeric film.

11. The system of claim 9 wherein determining one or more compressible and resilient consumable products which will fit within at least one of the one or more packable spaces comprises determining one or more compressible and resilient consumable products for which each dimension is equal to or smaller than a corresponding dimension of one of the one or more packable spaces.

12. The system of claim 9 wherein each of said compressible and resilient consumable products on said list of items can be compressed in at least one dimensional direction without damage to the product or packaging material surrounding the product to define a compressed dimension.

13. The system of claim 12 wherein determining one or more compressible and resilient consumable products which will fit within at least one of the one or more packable spaces comprises determining a compressible and resilient consumable product for which (1) first and second dimensions are collectively equal to or smaller than corresponding first and second dimensions of one of the one or more packable spaces; (2) a third dimension is greater than the corresponding third dimension of the one or more packable spaces; and (3) a minimum compressed third dimension is equal to or smaller than a corresponding third dimension of one of the one or more packable spaces.

14. The system of claim 9 wherein the computer program instructions are further configured for displaying a quantity of conventional fill material avoided by the customer's purchase of the one or more compressible and resilient consumable products.

15. The system of claim 9 wherein the system further includes generating instructions to a human or a robotic machine for filling the shipping container.

16. The system of claim 9 wherein a plurality of compressible and resilient products in the list of items are sized so that at least two dimensions are within 5 percent of two inner dimensions, respectively, of a shipping container commonly used by an on-line retailer.
